(19) United States
(12) Patent Application Publication
(10) Pub. No.: US 2026/0234093 A1
(43) Pub. Date: Aug. 13, 2026

(54) METHOD FOR PRODUCING BIOBASED a,B-UNSATURATED CARBOXYLIC ACIDS FROM POLY(3-HYDROXYALKANOATE) CONTAINED IN BIOMASS

(71) Applicant: Arkema France, Puteaux (FR)

(72) Inventors: Serge TRETJAK, Saint Avold Cedex (FR); Yves CABON, Saint Avold Cedex (FR)

(21) Appl. No.: 19/471,849

(22) PCT Filed: Mar. 29, 2024
(86) PCT No.: PCT/FR2024/050411

§ 371 Date: Oct. 2, 2025

(30) Foreign Application Priority Data
Apr. 5, 2023 (FR) 2303386

(51) Int. Cl. C07C 51/09; C07C 51/42

(52) U.S. Cl. CPC C07C 51/09; C07C 51/42

(57) ABSTRACT

The present invention relates to a process for producing biobased α,β-unsaturated carboxylic acids from a biomass containing a poly(3-hydroxyalkanoate), comprising the extraction of said poly(3-hydroxyalkanoate), followed by the thermolysis of said polymer in the solid or molten state, in the presence of polymerization inhibitors and in the absence of catalyst, leading to the production of biobased α,β-unsaturated carboxylic acids.

### TECHNICAL FIELD

The present invention relates to a process for producing biobased α,β-unsaturated carboxylic acids from a biomass containing a poly(3-hydroxyalkanoate), comprising the extraction of said poly(3-hydroxyalkanoate) in the presence of polymerization inhibitors, followed by a thermolysis of said polymer in the solid or molten state, in the absence of catalyst, leading to the production of biobased α,β-unsaturated carboxylic acids.

### PRIOR ART AND TECHNICAL PROBLEM

α,β-Unsaturated carboxylic acids are nowadays produced industrially mainly from feedstocks of fossil origin. For example, acrylic acid is obtained by oxidation of propylene, or methacrylic acid can be obtained by oxidation of isobutylene.

One possible way of obtaining these α,β-unsaturated carboxylic acids is the thermolysis, at temperatures of 150° C. to 300° C., of the corresponding poly(3-hydroxyalkanoate) (P3HA), according to the following reaction:

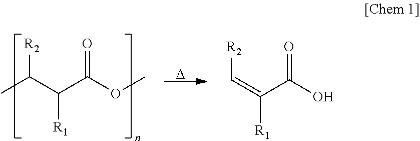

R₁=H or alkyl and R₂=H or alkyl; n is a number greater than 30

If R₁=R₂=H:
Poly(3-hydroxyalkanoate)=poly(3-hydroxypropionate) (P3HP);
α,β-Unsaturated carboxylic acid=propenoic acid (acrylic acid).

If R₁=methyl and R₂=H:
Poly(3-hydroxyalkanoate)=poly(3-hydroxyisobutyrate) (P3HiB);
α,β-Unsaturated carboxylic acid=isobutenoic acid (methacrylic acid).

If R₁=H and R₂=methyl:
Poly(3-hydroxyalkanoate)=poly(3-hydroxybutyrate) (P3HB);
α,β-Unsaturated carboxylic acid=but-2-enoic acid (crotonic acid).

If R₁=H and R₂=ethyl:
Poly(3-hydroxyalkanoate) is poly(3-hydroxyvalerate) (P3HV);
α,β-Unsaturated carboxylic acid=pent-2-enoic acid

These poly(3-hydroxyalkanoates) can themselves be obtained beforehand by chemical transformations of feedstocks of fossil origin, but also by fermentation of biomass.

There is strong market demand for these α,β-unsaturated carboxylic acids, which are used as monomers in numerous applications, to be obtained from biobased feedstocks. These biobased feedstocks are derived from renewable organic matter (biomass) of biological origin (microorganisms, plants or animals).

A potential problem with such a process is that the P3HA obtained by fermentation is present inside the cell. Thermolysis is therefore carried out in the presence of the cell membrane, which poses problems of fouling of the reactor or the presence of impurities in the end product.

A number of solutions to this problem have been proposed.

Document U.S. Pat. No. 9,850,192 describes a process for producing acrylic acid from a genetically modified microbial biomass metabolizing glucose or any other renewable feedstock to produce a poly(3-hydroxypropionate) (P3HP) homopolymer or copolymer inside microbial cells. Said process comprises a step of thermolysis of the washed/dried/milled P3HP-containing biomass in the presence of a catalyst. This process actually makes it possible to produce acrylic acid while limiting the formation of acrylic acid oligomers, such as the acrylic acid dimer which is spontaneously formed during the production of acrylic acid. Acrylic acid is recovered in gaseous form and then condensed, while the catalyst and the residual mass of biomass can be recycled into the process or subjected to thermal regeneration. However, there is a risk that the residue present in the reactor after thermolysis will be pasty and tacky, which could complicate its transfer to an industrial scale. Example 5 and FIG. 7 describe how to carry out this invention on an industrial scale. After fermentation, the biomass is washed, and dried using either an atomizer or a double drum dryer. After addition of the catalyst, the product is pyrolyzed in a FAST™ reactor at 250-350° C. with a residence time of between 0.25-1 hour using an inert gas such as nitrogen to send the vapors formed to the purification equipment. The vapor phase is composed of 90% organic/water and 10% inert gas. The gas is then purified, according to the process described in document U.S. Pat. No. 6,646,161 or in document US 20120006673, to obtain acrylic acid still containing many impurities. Complete purification is carried out using distillation columns, as described in documents U.S. Pat. Nos. 7,332,624 and 7,179,875, and may also require crystallization operations, as described in documents U.S. Pat. Nos. 6,482,981 and 7,179,875.

Another solution consists in first extracting the P3HA from the biomass using an organic solvent before subjecting it to thermolysis. Document US 20150376152 describes in example 6 the extraction of P3HP from biomass, using an organic solvent, such as 2-butanone, then the production of acrylic acid in three steps: evaporation of the solvent and condensation of the latter in a receiving vessel; thermal degradation of the P3HP in the absence of inhibitor leading to the production of acrylic acid vapor, and finally distillation and condensation of the acrylic acid in a receiving vessel containing hydroquinone to prevent the polymerization of the acrylic acid.

In their application FR 2208914, the applicant company proposes to carry out the thermolysis of the P3HA in the absence of catalyst and in the presence of a polymerization inhibitor; characteristically, the vapor pressure of at least one of the inhibitors at the thermolysis temperature is at least twice the pressure at which thermolysis is carried out, which has the effect of preventing the formation of polymers in the reactor and also the gas phase in the event of accidental condensation or at the time the acrylic acid vapors condense at the top of the column.

In their application FR 2208916, the applicant company describes a process using a solvent which makes it possible, starting from biomass, to selectively solubilize the P3HA, to separate the organic detritus insoluble in said solvent, then to carry out a thermolysis treatment of the P3HA and solvent medium in the liquid phase in the presence of polymerization inhibitors.

Document WO 2016/039618 describes the thermal degradation of a dry biomass containing poly(3-hydroxybutyrate) in order to produce crotonic acid. In example 1, it is shown that crotonic acid can be obtained with comparable yields of less than 60% from wet or dry biomass, in the absence of catalyst.

It has now been discovered that it is possible to simplify the procedure for producing α,β-unsaturated carboxylic acids, by carrying out the thermolysis of P3HA in the absence of solvent, after prior extraction of the P3HA from the cell membrane, and as a mixture with at least one solid-phase polymerization inhibitor, without using a catalyst and without injecting inert gas to entrain the vapors out of the reaction zone.

More precisely, “in the absence of catalyst” is understood to mean that the thermolysis of the PHA in the presence of at least one polymerization inhibitor takes place in the absence of another chemical species that accelerates or reorients the reaction kinetics. The thermolysis as envisaged is induced only by operating conditions such as temperature, pressure and residence time.

Actually excluded are the chemical species that lead to different types of catalysis depending on the nature of the species:
homogeneous catalysis, if the catalyst and the reactants form only one phase (often a liquid phase);
heterogeneous catalysis, if the catalyst and the reactants form several phases (usually a solid catalyst for gas-phase or liquid-phase reactants);
enzymatic catalysis, if the catalyst is an enzyme, i.e. a protein.

The invention accordingly proposes to provide a simple and easily implementable solution for reducing the fouling phenomena and the presence of impurities in the end product and thus maintain high reliability and increased productivity in processes for producing α,β-unsaturated carboxylic acids from poly(3-hydroxyalkanoates) obtained by fermentation.

### SUMMARY OF THE INVENTION

The present invention relates to a process for producing a biobased α,β-unsaturated carboxylic acid from a biomass containing a poly(3-hydroxyalkanoate) (P3HA) in the absence of catalyst, said process comprising the following steps:
extracting the poly(3-hydroxyalkanoate) from the biomass using a solvent capable of solubilizing the P3HA;
evaporating off the solvent to give a solid P3HA having a purity of at least 95% by weight;
mixing the extracted P3HA with at least one polymerization inhibitor in the solid state;
subjecting said P3HA-inhibitor solid mixture to a thermolysis step resulting in, firstly, said α,β-unsaturated carboxylic acid in the vapor phase and, secondly, a molten residue;
separating the two phases formed into a gas phase and a solid phase;
purifying said gas phase to obtain a purified α,β-unsaturated carboxylic acid;
treating the solid-phase residue.

According to various implementations, said process comprises the following features, where appropriate in combination. The contents indicated are expressed by weight, unless otherwise mentioned. In the ranges of values indicated, the limits are included.

According to one embodiment, the poly(3-hydroxyalkanoate) used in the process comprises a single type of 3-hydroxyalkanoate unit and the product formed is therefore composed of a single α,β-unsaturated carboxylic acid.

According to one embodiment, the poly(3-hydroxyalkanoate) is poly(3-hydroxypropionate) and the α,β-unsaturated carboxylic acid produced is acrylic acid.

According to one embodiment, the poly(3-hydroxyalkanoate) is poly(3-hydroxyisobutyrate) and the α,β-unsaturated carboxylic acid produced is methacrylic acid.

According to one embodiment, the poly(3-hydroxyalkanoate) is poly(3-hydroxybutyrate) and the α,β-unsaturated carboxylic acid produced is crotonic acid.

According to one embodiment, the poly(3-hydroxyalkanoate) used in the process comprises a plurality of different 3-hydroxyalkanoate units and the product formed is therefore composed of a mixture of different α,β-unsaturated carboxylic acids. Examples of P3HA copolymers are poly-3-hydroxybutyrate-co-3-hydroxypropionate (poly-3HB-co-3HP) or poly-3-hydroxybutyrate-co-3-hydroxyvalerate (poly-3HB-co-3HV).

According to one embodiment, the poly(3-hydroxyalkanoate) contains the 3-hydroxypropionate unit and at least one of the α,β-unsaturated carboxylic acids produced is acrylic acid.

According to one embodiment, the poly(3-hydroxyalkanoate) contains the 3-hydroxyisobutyrate unit and at least one of the α,β-unsaturated carboxylic acids produced is methacrylic acid.

According to one embodiment, the poly(3-hydroxyalkanoate) contains the 3-hydroxybutyrate unit and at least one of the α,β-unsaturated carboxylic acids produced is crotonic acid.

According to one embodiment, the biomass host is a bacterium, yeast, fungus, alga, cyanobacterium or a mixture of two or more of these elements.

Depending on the embodiment, the P3HA used is extracted beforehand from the biomass, to yield a solid P3HA having a purity of at least 95%.

According to one embodiment, the process according to the invention comprises a step of condensing the vapor of the α,β-unsaturated carboxylic acid(s) obtained by the thermolysis reaction of poly(3-hydroxyalkanoate), followed by one or more purification steps. The purification operations can generally include distillations, liquid/liquid extractions, separations using a film evaporator, or crystallizations.

According to one embodiment, the process according to the invention comprises a step of treating said molten residue obtained at the end of thermolysis, for example by upgrading the latter via hydrothermal gasification to give methane.

The present invention meets the need expressed in the prior art. It makes it possible to prevent the risks of fouling of the thermolysis reactor and/or presence of impurities in the α,β-unsaturated carboxylic acids end product, originating from cell membranes, by making it possible to obtain a gas phase rich in α,β-unsaturated carboxylic acid and also containing polymerization inhibitors and also a molten or slightly pasty residue that can be upgraded.

This solution has the advantage of performing the thermolysis of the P3HA in the solid or molten state, which reduces the energy and environmental cost of the process.

The invention will now be described in more detail in the description that follows.

### DETAILED DESCRIPTION OF THE INVENTION

The aim of the invention is to produce biobased α,β-unsaturated carboxylic acids on an industrial scale by thermolysis of poly(3-hydroxyalkanoate) contained in the biomass, while limiting problems with clogging of the thermolysis reactor and/or with the presence of impurities in the end product.

The term “thermolysis” of poly(3-hydroxyalkanoate) (P3HA) means its chemical decomposition into α,β-unsaturated carboxylic acid that occurs under the effect of temperature. This term is synonymous with pyrolysis.

The present invention relates to a process for producing a biobased α,β-unsaturated carboxylic acid from a biomass containing a poly(3-hydroxyalkanoate) (P3HA) and in the absence of catalyst, said process comprising the following steps:
extracting the poly(3-hydroxyalkanoate) from the biomass using a solvent capable of solubilizing the P3HA;
evaporating off the solvent to give a solid P3HA having a purity of at least 95% by weight;
mixing the extracted P3HA with at least one polymerization inhibitor in the solid state;
subjecting said P3HA-inhibitor solid mixture to a thermolysis step resulting in, firstly, said α,β-unsaturated carboxylic acid in the vapor phase and, secondly, a molten residue;
separating the two phases formed into a gas phase and a solid phase;
purifying said gas phase to obtain a purified α,β-unsaturated carboxylic acid;
treating the solid-phase residue.

According to one embodiment, said process for producing a biobased α,β-unsaturated carboxylic acid from a biomass containing a poly(3-hydroxyalkanoate) (P3HA) comprises the following steps:
extracting the poly(3-hydroxyalkanoate) from the biomass using a solvent capable of solubilizing the P3HA in a solvolysis reactor;
removing the cell membrane by liquid-solid separation;
evaporating off the solvent to give a solid P3HA having a purity of at least 95% by weight in this same reactor;
introducing the P3HA and at least one polymerization inhibitor into a thermolysis reactor;
mixing the P3HA and at least one polymerization inhibitor in said reactor;
thermolyzing this stirred mixture at a given temperature and at a controlled pressure in this same reactor in order to generate a vapor phase and a viscous phase;
separating the two phases formed in a gas-liquid separator, resulting in a gas phase and a residue;
treating said residue;
condensing the gas phase;
treating the condensed phase to obtain the α,β-unsaturated carboxylic acid by using one to several distillation columns making it possible, firstly, to separate the α,β-unsaturated carboxylic acid from the products heavier than the latter and, secondly, to obtain products lighter than the latter.

According to one embodiment, said solvolysis reactor is stirred and heated to a temperature of between 20° C. and 170° C., preferably 50° C. to 140° C.

According to one embodiment, the cell membrane is removed by filtration or centrifugation.

According to one embodiment, the solvent is evaporated off by heating under reduced pressure of between 3 kPa and 101 kPa, preferably between 20 kPa and 60 kPa, in a temperature range between 50° C. and 140° C.

According to one embodiment, the introduction of the P3HA and at least one polymerization inhibitor into said thermolysis reactor is carried out by means of a pipe or by a conveyor of the endless screw type.

Advantageously, said thermolysis reactor is suitable for the treatment of solid, molten or pasty mixtures.

According to one embodiment, the mixing of the P3HA and at least one polymerization inhibitor in said thermolysis reactor is carried out by means of several endless screws actuated in a barrel allowing the mixing of the P3HA and at least one inhibitor.

According to one embodiment, said residue is treated by upgrading by land spreading, combustion or hydrothermal gasification.

According to one embodiment, the condensation of the gas phase is carried out by a system of at least one condenser of the tubular condenser type at the pressure of the thermolysis, by cooling it and collecting the liquid phase obtained in a stirred storage tank, optionally with the addition of one or more additional inhibitors.

According to one embodiment, said products heavier than the α,β-unsaturated carboxylic acid are recycled upstream of the thermolysis reactor, or are mixed with the solid residue resulting from the thermolysis.

According to one embodiment, said products lighter than the α,β-unsaturated carboxylic acid are upgraded by combustion or hydrothermal gasification.

According to one embodiment, the α,β-unsaturated carboxylic acid obtained is purified by a fractional crystallization operation comprising several separation stages to obtain high purity α,β-unsaturated carboxylic acid and a residue to be upgraded as energy or recycled.

According to one embodiment, the process according to the invention makes it possible to manufacture several biobased α,β-unsaturated carboxylic acids from the poly(3-hydroxyalkanoate)s contained in the biomass.

The invention is based on the use of a mixture of P3HA and at least one polymerization inhibitor by performing a technique for mixing solids and heat treating this mixture.

The term “biomass” means organic matter originating from plants (including microalgae), animals, bacteria or fungi that is employable as a source of biobased feedstocks, as opposed to feedstocks of fossil origin.

In the process according to the invention, the first step uses genetically modified host biomass derived from genetic engineering. According to one embodiment, the biomass host is a bacterium, yeast, fungus, alga, cyanobacterium or a mixture of two or more of these elements.

The biomass is obtained by a prior step of culturing a recombinant host with a renewable feedstock. According to one embodiment, the renewable feedstock is selected from glucose, fructose, sucrose, arabinose, maltose, lactose, xylose, ethanol, methanol, glycerol, fatty acids, vegetable oils and biomass-derived synthesis gas or a combination thereof.

According to one embodiment, the biomass used in the process according to the invention comes from a process of bacterial fermentation of sugars or lipids.

Depending on the culture conditions and on the variety of microorganism used, poly(3-hydroxyalkanoate) (P3HA) homopolymers or copolymers with different 3-hydroxyalkanoic acid units are formed.

The biomass used is pretreated beforehand by means of washing, drying or milling operations to yield a biomass containing at least 30% by weight of P3HA, preferably at least 50% by weight of P3HA.

The step of extracting the P3HA from the biomass with a solvent comprises a separation of organic waste insoluble in said solvent, for example the cell membranes, from the P3HA-solvent mixture, which is carried out by filtration or by centrifugation.

The step of extracting the P3HA from the biomass with a solvent takes place at a temperature of 20° C. to 130° C.

According to one embodiment, the step of extracting the P3HA from the biomass with a solvent takes place batchwise.

According to a preferred embodiment, the step of extracting the P3HA from the biomass with a solvent takes place continuously.

According to one embodiment, the solvent used to extract the P3HA present in the biomass at atmospheric pressure is selected from polar solvents having a boiling point higher than the extraction temperature but lower than the thermolysis temperature. These solvents may be linear or branched alcohols with a carbon number of less than or equal to 7, for example heptanol or n-butanol, linear or branched aldehydes or ketones with a carbon number of less than or equal to 7, such as hexanal or butanone, or carboxylic acids with a carbon number lower than C4, such as butyric acid.

The solvent used in the process must be capable of solubilizing the P3HA in a content of greater than 5% by weight in the solution, preferably greater than 20%, at the temperature used during the extraction step.

The solvent is then evaporated off to give a solid P3HA having a purity of at least 95% by weight.

Depending on the embodiment, the evaporation can be carried out under a pressure of 20 kPa to 100 kPa at a temperature of between 20° C. and 150° C.

The solid P3HA is then mixed with at least one polymerization inhibitor chosen from the inhibitors conventionally used in existing industrial processes for the production of α,β-unsaturated carboxylic acids. These include phenol derivatives such as hydroquinone (HQ) and derivatives thereof such as hydroquinone methyl ether (MEHQ), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

According to a preferred embodiment, at least one of said polymerization inhibitors is hydroquinone methyl ether (MEHQ).

According to one embodiment, the weight content of inhibitor in the mixture with the P3HA is between 0.1% and 10%, preferably from 0.4% to 5%.

According to the invention, the system for feeding the thermolysis reactor with P3HA and at least one polymerization inhibitor may be a pipe, an endless screw, a conveyor belt or a hopper, a pneumatic conveyor, a vibrating conveyor or an extruder. In addition, they may be coupled to a metering device.

The mixture of P3HA and at least one polymerization inhibitor is then subjected to thermolysis in the solid or molten state.

The step of mixing the P3HA and at least one polymerization inhibitor can be carried out in a mixer-conveyor comprising several endless screws actuated in a barrel, or directly in the thermolysis reactor.

According to one embodiment, the mixing and thermolysis steps are carried out continuously by successively performing the conveyor mixing and the thermolysis reaction or by performing these two operations in the thermolysis reactor.

Preferably, the thermolysis reactor carries out the mixing and the thermolysis reaction.

Heating of the mixture can be carried out at a temperature between 100° C. and a temperature lower than the self-ignition temperature of the monomer formed. For acrylic acid, this temperature is 438° C. at atmospheric pressure (NF T20037 standard). Preferably, the heating temperature is between 150° C. and 400° C., advantageously between 200° C. and 350° C. The heating can be staged, with a first temperature zone of the order of 100° C.-200° C., which makes it possible to liquefy all or part of the mixture while avoiding the polymerization of the acrylic acid.

According to one embodiment, the pressure in the thermolysis reactor is between 3 kPa and 101 kPa, preferably between 15 kPa and 40 kPa.

According to one embodiment, the residence time in the thermolysis reactor is between 0.05 h and 1 h, preferably between from 0.15 h to 0.5 h.

According to one embodiment, the apparatus for carrying out the process also comprises a reactor suitable for the heating for the purpose of the thermolysis. For example, the heating can be carried out by exposing the mixture to microwaves, to pulsed electric fields or to a preheated inert gas or steam, with a preheated solid such as sand, by contact with a hot surface for instance in an extruder, a screw conveyor, a rotating drum or a platen. The hot surface can be heated by various means: direct electric heating, heating by heat transfer fluid (steam, oil, molten salts).

According to one embodiment, the heat is supplied through a hot surface heated by a heat-transfer fluid and in particular molten salts.

The thermolysis reactor according to the invention may be an extruder or conveyor, a reactor suitable for pyrolysis, for high-temperature pyrolysis, or a fluidized reactor or a reactor suitable for solvolysis or else a reactor consisting of hollow plates heated by a heat-transfer fluid circulating in the plates. However, reactors enabling higher gains in yield of α,β-unsaturated carboxylic acid have been identified, such as: a conveyor, an extruder, an extruder-conveyor and/or a set of heating plates.

According to the invention, the extruder-conveyor is a reactor comprising one or more endless screws each actuated in a barrel, notably allowing the blending of the components introduced into said barrel. The use of an extruder-conveyor for performing this P3HA thermolysis process is advantageous from an environmental, security and safety viewpoint of the process. Specifically, an extruder-conveyor makes it possible to treat a molten medium without the need to add solvent to reduce the viscosity of the molten medium. The extruder-conveyor has the advantage of allowing efficient heat transfer from the barrel to the P3HA-inhibitor medium. The extruder may advantageously be replaced with a screw conveyor system over all or part of the length thereof. Advantageously, the system may comprise the combination of a conveyor-type device in the first part, followed by an extruder-type device and by a conveyor configured to transport the residue to the outlet. For example, the conveyor can be of the “Archimedes screw” (endless screw) type.

A thermolysis system according to the invention may comprise an extruder, such as the twin-screw extruder 200, comprising an inlet for the solid or previously melted P3HA-inhibitor mixture. A twin-screw extruder may be a Clextral extruder. The twin-screw extruder comprises two screws, which are usually parallel, rotating inside a barrel. Advantageously, the extruder is of modulable nature, i.e. the screw and the barrel are modules assembled in series, and the assembly of which may be modified. In the extruder, an external heating means regulating the temperature of the barrel is advantageously configured to, on the one hand, bring the P3HA-inhibitor mixture to the molten state, and, on the other hand, carry out the thermolysis of the P3HA.

According to another embodiment, the thermolysis system comprises a device consisting of hollow plates, heated by a heat-transfer fluid (pressurized steam, oil, molten salts, etc.) circuit. In the course of its treatment, the article firstly advances over plates with increasing temperatures. The residue ends its passage through the reactor by passing over plates which are at a lower temperature and where the heat exchange takes place from the residue to the heat-transfer fluid. The heat transfer fluid thus heated can then be used to preheat the P3HA-inhibitor mixture feeding the thermolysis reactor.

According to one embodiment, the thermolysis system is a device of mixer-conveyor type, for example of screw conveyor type. This device comprises a reactor in which two endless screws operate in opposition. The mixture is heated through the hot wall using a heat-transfer fluid such as steam. The movement of the two screws makes it possible to mix and homogenize the feeds of P3HA and polymerization inhibitor.

According to one embodiment, the process according to the invention carries out thermolysis of the P3HA-inhibitor mixture by means of an extruder at a temperature of the order of 150-400° C.

In the thermolysis reactor, the P3HA-inhibitor mixture is converted under the action of heat into gaseous compounds comprising an α,β-unsaturated carboxylic acid.

According to one embodiment, the poly(3-hydroxyalkanoate) is poly(3-hydroxypropionate) (P3HP), and the α,β-unsaturated carboxylic acid obtained by the process according to the invention is acrylic acid.

According to one embodiment, the poly(3-hydroxyalkanoate) is poly(3-hydroxyisobutanoate) (P3HiB), and the α,β-unsaturated carboxylic acid obtained by the process according to the invention is methacrylic acid.

According to one embodiment, the poly(3-hydroxyalkanoate) is poly(3-hydroxybutanoate) (P3HB), and the α,β-unsaturated carboxylic acid obtained by the process according to the invention is crotonic acid.

According to one embodiment, the invention relates to a process for producing a mixture of α,β-unsaturated carboxylic acids from a P3HA contained in the biomass, comprising a plurality of different 3-hydroxyalkanoate units.

Gases containing the α,β-unsaturated carboxylic acid(s) can be sent to a cooling system in order to be condensed. The condensate obtained may then be collected in a chamber intended for this purpose. The reactor enclosure and also the chamber are preferably under reduced pressure. The condensation system may be equipped with an injection of one or more inhibitors. In order to enable the recovery of a purified α,β-unsaturated carboxylic acid, the system may comprise a purification device, for example one or more distillation columns, or one or more items of liquid extraction, crystallization or membrane separation equipment.

The solid residue is then upgraded, for example by hydrothermal gasification or in the form of a fuel.

According to one embodiment, condensation is carried out using one or more tubular or spiral exchangers in series.

According to one embodiment, condensation is carried out by successive pressure adjustment and separation of the resulting gas and liquid phases containing α,β-unsaturated carboxylic acid and contaminants which can be recycled back into the reactor.

According to the invention, the condensation is carried out by a successive condensation temperature adjustment carried out by placing one or more condensers in series and separation of the resulting gas and liquid phases containing α,β-unsaturated carboxylic acid and contaminants which can be recycled back into the reactor or sent to the purification system.

According to one embodiment, one or more polymerization inhibitors are added to the condenser.

According to one embodiment, this condensation can be carried out by bringing the α,β-unsaturated carboxylic acid in the gaseous state into contact with the α,β-unsaturated carboxylic acid in the liquid state. This contacting operation may be performed, for example, in a device of shower type, by spraying the liquid α,β-unsaturated carboxylic acid into a chamber collecting the α,β-unsaturated carboxylic acid in the gaseous state.

According to one embodiment, there is no addition of inhibitor to the condenser.

According to one embodiment, the residue obtained after the thermolysis step is upgraded by hydrothermal gasification.

According to the embodiment, the hydrothermal gasification is carried out at a temperature of 350° C.-450° C. and a pressure of 25 MPa.

The examples below illustrate the present invention without, however, limiting the scope thereof.

### EXPERIMENTAL SECTION

The examples are carried out on biomass containing 60% by weight of poly(3-hydroxypropionate) (P3HP). The α,β-unsaturated carboxylic acid obtained after thermolysis is acrylic acid (AA).

The biomass containing the P3HP is brought into contact with a solvent solubilizing the P3HP, then this mixture is treated by centrifugation in order to separate the insoluble materials (for instance the cell membrane) from the P3HP-solvent mixture. The solvent is then evaporated under vacuum in order to recover the solid P3HP.

The thermolysis is carried out by placing the solid P3HP (2 g) and the inhibitor (0 or 20 mg of MEHQ or PTZ) in a 50-ml two-neck round-bottom flask equipped with a bar magnet. The medium is stirred using a magnetic stirrer, so as to spread the inhibitor throughout the solid. This 50-ml two-neck round-bottom flask containing the medium is equipped on the side neck with a thermometer to monitor the temperature of the thermolysis medium and on the upper neck with a separation bridge leading to a water-cooled side condenser. The condenser leads to a receiver consisting of a 25-ml single-neck round-bottom flask. An air bleed allows the experiment to be performed under a partial vacuum.

At the start of the experiment, the system is placed under the desired pressure, and the round-bottom flask containing the P3HP-inhibitor mixture is then placed in a heating system allowing the desired thermolysis temperature to be established (oil bath or electric heating mantle). The receiver is cooled by an ice bath.

As soon as the thermolysis reactor reaches more than 170° C., AA vapors are formed, condensing predominantly in the side condenser. After 4 h of heating, the formation of AA vapors in the thermolysis reactor tapers off and the experiment is then stopped. The consistency of the thermolysis residue is assessed visually at the end of the experiment.

The results obtained are presented in table 1.

TABLE 1

| | | Vapor | | |
|---|---|---|---|---|
| | | pressure of | | |
| | | the inhibitor | Operating | |
| | | at 200° C. | pressure | |
| Test | Inhibitor | (kPa) | (kPa) | Thermolysis residue |
| 1 | No | — | 100 | Tacky hard solid |
| 2 | No | — | 55 | Tacky hard solid |
| 3 | No | — | 20 | Tacky hard solid |
| 4 | MEHQ | 28.5 | 100 | Viscous pasty solid |
| 5 | MEHQ | 28.5 | 55 | Viscous pasty solid |
| 6 | MEHQ | 28.5 | 20 | Viscous pasty solid |
| 7 | PTZ | 0.7 | 100 | Tacky pasty solid |
| 8 | PTZ | 0.7 | 55 | Tacky pasty solid |
| 9 | PTZ | 0.7 | 200 | Tacky pasty solid |

The results in table 1 demonstrate that the physical state of the residue is dependent on the presence of the inhibitor, in particular of MEHQ. The addition of inhibitor enables the residue to become pasty and viscous, whereas it was tacky without the addition of the latter. This change in consistency of the residue allows easier extraction of the latter when it comes to carrying out the thermolysis continuously.

The tests of examples 10-12 below are carried out in the same laboratory assembly.—using P3HP purified from biomass as described above.

### Example 10 (Comparative): Use of Pure P3HP without Catalyst and without Inhibitor

2.05 g of purified P3HP are placed in a 25-ml two-neck round-bottom flask equipped with magnetic stirring. The round-bottom flask is placed at 20 kPa of pressure by means of a diaphragm vacuum pump and then heated at 200° C. for 4 h. The vapors generated are condensed using a water-cooled side condenser to obtain 1.41 g of acrylic acid, which corresponds to 68% yield. The solid obtained after cracking forms a thin layer which remains stuck to the walls of the two-necked round-bottom flask. The layer formed is very difficult to remove from the two-neck round-bottom flask. Solid particles are found in the top of the flask and on the side condenser.

In the absence of a catalyst, the acrylic acid recovery yield is low, of the order of 68%. This low value is consistent with that cited in document WO 2016/039618, example 1 in which thermolysis of PHB leads to the production of 57% crotonic acid. Added to this low yield is the presence of solid particles prejudicial to the development of this process.

### Example 11 (According to the Invention): Use of Pure P3HP with Addition of 1% of 4-Methoxyphenol (MEHO) without Catalyst

2.12 g of purified P3HP are placed in a 25-mi two-neck round-bottom flask equipped with magnetic stirring. 0.021 g of MEHQ are added to the round-bottom flask and mixed with the PH3P. The round-bottom flask equipped with a separation bridge is placed at 20 kPa of pressure by means of a diaphragm vacuum pump. The flask is heated at 200° C. for 4 h. The vapors generated are condensed using a water-cooled side condenser to obtain 1.99 g of acrylic acid, which corresponds to 94% yield. After cracking, very little solid remains in the round-bottom flask, this solid is easily removed from the round-bottom flask by simple scraping.

### Example 12 (According to the Invention): Use of Pure P3HP with Addition of 5% of 4-Methoxyphenol (MEHO) without Catalyst

2.12 g of purified P3HP are placed in a 25-ml two-neck round-bottom flask equipped with magnetic stirring. 0.117 g of MEHQ are added to the round-bottom flask and mixed with the PH3P. The round-bottom flask equipped with a separation bridge is placed at 20 kPa of pressure by means of a diaphragm vacuum pump. The flask is heated at 200° C. for 4 h. The vapors generated are condensed using a water-cooled side condenser to obtain 1.85 g of acrylic acid, which corresponds to 87% yield. After cracking, very little solid remains in the round-bottom flask, this solid is easily removed from the round-bottom flask by simple scraping.

The yield obtained in examples 11 and 12 is therefore much higher than that reported in example 1 of document WO 2016/039618, where the thermal degradation also takes place in the absence of catalyst. It is only when the thermolysis is carried out in the presence of catalyst that the yield increases to 86% or 89% respectively (for examples 2 and 3 of said document).

These examples also show that in the presence of an inhibitor, in the process according to the invention, the thermolysis yields are very high in the absence of catalyst and even under more moderate thermal conditions since this thermolysis is carried out at 200° C. (290° C. with catalyst in WO 2016/039618, example 1).

1. A process for producing a biobased α,β-unsaturated carboxylic acid from a biomass containing a poly(3-hydroxyalkanoate) (P3HA) and in the absence of catalyst, said process comprising the following steps:
extracting the poly(3-hydroxyalkanoate) from the biomass using a solvent capable of solubilizing the P3HA;
evaporating off the solvent to obtain a solid P3HA having a purity of at least 95% by weight;
mixing the solid P3HA with at least one polymerization inhibitor in the solid state to produce a P3HA-inhibitor solid mixture;
subjecting said P3HA-inhibitor solid mixture to a thermolysis step resulting in said α,β-unsaturated carboxylic acid in the vapor phase and a molten residue;
separating the α,β-unsaturated carboxylic acid into a gas phase and the molten residue into a solid phase residue;
purifying said gas phase to obtain a purified α,β-unsaturated carboxylic acid; and
treating the solid-phase-residue.

2. The process as claimed in claim 1, wherein:
extracting the poly(3-hydroxyalkanoate) from the biomass using a solvent capable of solubilizing the P3HA is carried out in a solvolysis reactor, wherein a cell membrane is removed by liquid-solid separation;
evaporating off the solvent to obtain the solid P3HA having a purity of at least 95% by weight is carried out in the solvolysis reactor;
introducing the P3HA and at least one polymerization inhibitor into a thermolysis reactor;
mixing the solid P3HA with the at least one polymerization inhibitor in a thermolysis reactor by introducing the P3HA and the at least one polymerization inhibitor into the thermolysis reactor;
subjecting said P3HA-inhibitor solid mixture to the thermolysis step in the thermolysis reactor or in another reactor of the same type to generate the gas phase and the molten residue;
separating the gas phase and the molten residue in a gas-liquid separator to form the gas phase and the solid phase residue;
the method further comprising:
condensing said gas phase; and
treating the condensed phase to obtain the α,β-unsaturated carboxylic acid by using one to several distillation columns, firstly, to separate the α,β-unsaturated carboxylic acid from the products heavier than the α,β-unsaturated carboxylic acid and, secondly, to obtain products lighter than the α,β-unsaturated carboxylic acid.

3. The process as claimed in claim 1, wherein the biomass is pretreated by means of washing, drying or milling operations to yield a biomass containing at least 30% by weight of P3HA.

4. The process as claimed in claim 1, wherein the poly(3-hydroxyalkanoate) comprises a 3-hydroxypropionate unit and at least one of the α,β-unsaturated carboxylic acids produced is acrylic acid.

5. The process as claimed in claim 1, wherein the poly(3-hydroxyalkanoate) is poly(3-hydroxypropionate) and the α,β-unsaturated carboxylic acid produced is acrylic acid.

6. The process as claimed in claim 1, wherein the poly(3-hydroxyalkanoate) comprises a 3-hydroxybutyrate unit and at least one of the α,β-unsaturated carboxylic acids produced is crotonic acid.

7. The process as claimed in claim 1, wherein the poly(3-hydroxyalkanoate) is poly(3-hydroxybutyrate) and the α,β-unsaturated carboxylic acid produced is crotonic acid.

8. The process as claimed in claim 1, wherein the poly(3-hydroxyalkanoate) comprises a 3-hydroxyisobutyrate unit and at least one of the α,β-unsaturated carboxylic acids produced is methacrylic acid.

9. The process as claimed in claim 1, wherein the poly(3-hydroxyalkanoate) is poly(3-hydroxyisobutyrate) and the α,β-unsaturated carboxylic acid produced is methacrylic acid.

10. The process as claimed in claim 1, wherein the at least one polymerization inhibitor is selected from phenol derivatives, phenothiazine derivatives, nitroxide derivatives or para-phenylenediamine derivatives.

11. The process as claimed in claim 1, wherein the at least one polymerization inhibitor is hydroquinone methyl ether.

12. The process as claimed in claim 1, wherein the solvent used to extract the P3HA present in the biomass is selected from polar solvents having a boiling point higher than an extraction temperature but lower than a thermolysis temperature.

13. The process as claimed in claim 2, wherein the thermolysis reactor is selected from: a conveyor, a mixer-conveyor, an extruder, an extruder-conveyor and/or a set of heating plates.

14. The process as claimed in claim 13, wherein the thermolysis reactor is an extruder-conveyor having one or more endless screws each actuated in a barrel.

15. The process as claimed in claim 13, wherein the thermolysis reactor is a twin-screw extruder.

16. The process as claimed in claim 13, wherein the thermolysis reactor is a device consisting of hollow plates heated by a heat-transfer fluid circuit.

17. The process as claimed in claim 13, wherein the thermolysis reactor is a screw conveyor.

18. The process as claimed in claim 1, wherein the thermolysis is carried out between 150° C. and 400° C. with a residence time of between 0.05 h and 1 h.

19. The process as claimed in claim 3, wherein the biomass contains at least 50% by weight of P3HA.
